(19) United States
(12) Patent Application Publication
(10) Pub. No.: US 2003/0048633 A1
(43) Pub. Date: Mar. 13, 2003

(54) Portable dual lamp set

(76) Inventors: Jong-Jiing Shiau, Taichung (TW); Chi-Te Su, Taichung (TW)

Correspondence Address: BACON & THOMAS, PLLC, 625 SLATERS LANE, FOURTH FLOOR, ALEXANDRIA, VA, 22314

(21) Appl. No.: 09/948,544

(22) Filed: Sep. 10, 2001

(51) Int. Cl. F21V 33/00

(52) U.S. Cl. 362/109

(57) ABSTRACT

A portable dual lamp set consists of a base frame and a lamp casing which houses a germicidal lamp and a black-glass lamp therein. The base frame has a pedestal and a frame wall bent and extended upwards to allow the base frame standing upright. The lamp casing may be separated from the base frame, or be coupled with the base frame and be stretched or tilted an angle to form different shapes for use. The lamp set has dual effects for inspection and sterilization.

### BACKGROUND OF THE INVENTION

The present invention relates to a lamp set and particularly a dual lamp set that has high portability for hand carrying or stationary mounting at different angles and is capable of providing dual effects for inspecting and sterilizing use.

Black-glass lamps containing tricolor rare earth elements as fluorescent agents for examining and testing purposes are commonly used elements in test instruments. The black-glass lamps can emit invisible ultraviolet light (300-400 nm) which may be used to inspect stains on machinery, clothes, and skins, and to examine documents, gems and counterfeit currency, and to illuminate stages and billboards.

Conventional germicidal lamps emit invisible ultraviolet light (254 nm) to kill bacteria in a short time and do not generate foul smell. They mostly are used for medical treatments or cosmetic purposes, and have become an essential sterilizing instrument in people's daily life.

Presently, the foregoing two products are classified as technology products and have been used in many different areas. When in use, they usually have to couple with huge facilities. It is not convenient and is expensive, therefore is not suitable for general household use. They also rarely are owned and used at the same time. Hence their benefits have not been fully utilized and enjoyed. There are still rooms for improvement.

### SUMMARY OF THE INVENTION

The primary object of the present invention is to provide a compact and portable dual lamp set that includes concurrently a black-glass lamp and a germicidal lamp and to offer inspection and sterilization effects at the same time.

To attain the foregoing objects, the dual lamp set of the present invention mainly includes a base frame with a lamp casing mounted thereon. The lamp casing houses a germicidal lamp and a black-glass lamp inside. The base frame has a pedestal and a frame wall bent and extended for standing upright. The lamp casing may be detached from the base frame, or be coupled, stretched, tilted an angle relative to the base frame to form different using conditions, and at the same time provides testing and sterilizing function.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is an exploded view of the present invention.

FIG. 2 is a perspective view of the present invention.

FIG. 3 is a schematic view of the present invention in use.

FIG. 4 is another schematic view of the present invention in use.

### DESCRIPTION OF THE PREFERRED EMBODIMENT

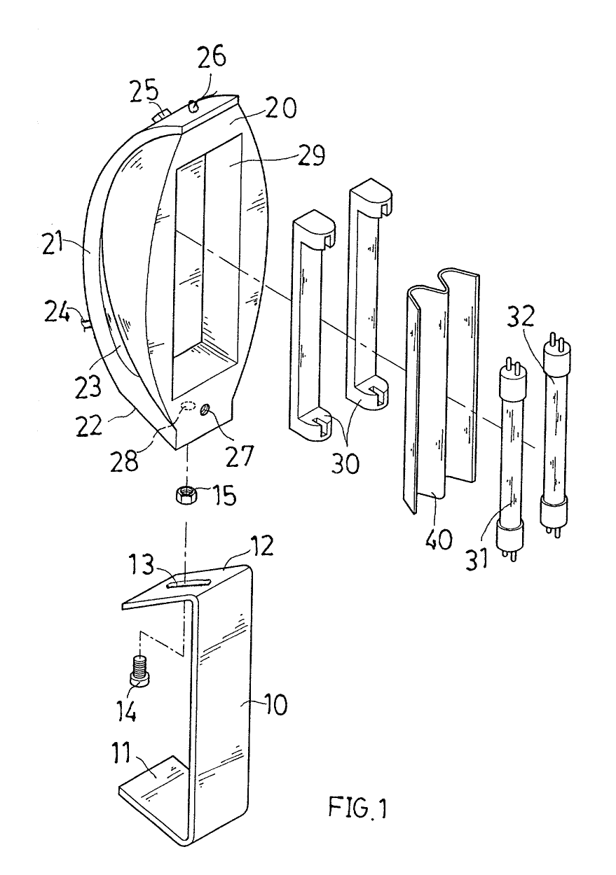

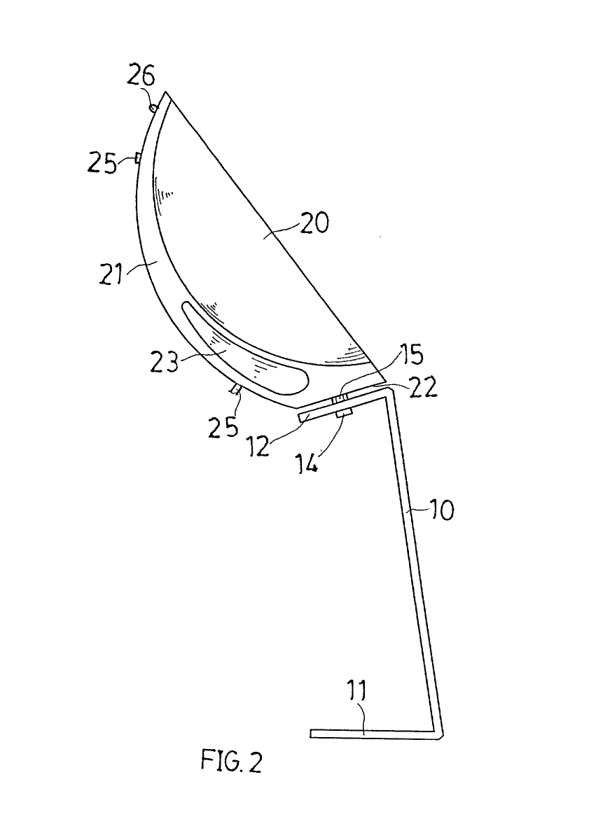

Referring to FIGS. 1 through 4, the present invention mainly consists of:

A base frame 10 which includes a pedestal 11 and a frame wall 12 bent and extended upwards to allow the base frame 10 standing upright. The frame wall 12 has two bordering side flanges 13 each has a border aperture 14 formed at an upper section proximate the top rim thereof. The side flange 13 around the border aperture 14 also has a plurality of positioning apertures 15 formed thereon. The frame wall 12 may be a transparent board or a colored transparent board;

A lamp casing 20 having a size may be held by users with hands includes two indented slide channels 21 located at two sides thereof to match the side flanges 13 of the base frame 10. In other words, the slide channels 21 are wedged on the side flanges 13, and allow the lamp casing 20 be separated from the base frame 10, or be coupled with the base frame 10 and be stretched to different shapes for use (operation details will be elaborated later). There is a power cord 23 linking to the periphery of the lamp casing 20 to provide power supply required, and a three-way switch 23, and an indication light 24 for displaying power supply conditions. Proximate the upper end of the slide channel 21, there is an anchoring means which includes a spring 25 and a steel ball 26 disposed sequentially in a cavity 27 which has a closed end such that the steel ball 26 may be urged in the positioning apertures 15 and border aperture 14. The lamp casing 20 further has a housing chamber 28 at one side;

Two lamp brackets 30 located in the housing chamber 28 of the lamp casing 20 for mounting respectively a germicidal lamp 31 and a black-glass lamp 32; and

A reflection hood 40 located between the lamp brackets 30 and the germicidal lamp 31 and black-glass lamp 32 to increase light intensity of the projecting light.

By means of the construction set forth above, the black-glass lamp 32 and the germicidal lamp 31 will generate respectively ultraviolet light of different wave lengths (300-400 nm, and 254 nm) to produce different effects desired. The black-glass lamp 32 may be used for detecting stains on machinery, clothes and skins, and for examining documents, gems, counterfeit currency or the like. The germicidal lamp 31 may be used for sterilization.

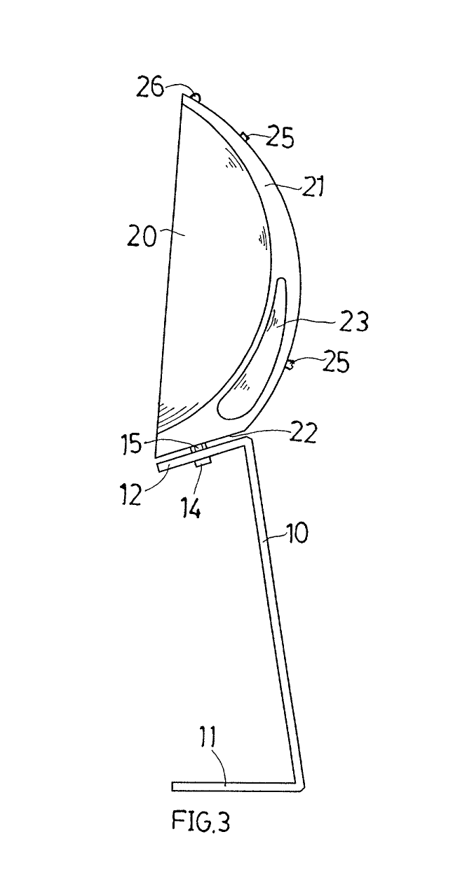

When in use, the slide channels 21 may be coupled with the side flanges 13, and allow the lamp casing 20 sliding relatively to the base frame 10, such as pulling the lamp casing 20 along the slide channels 21 upwards for a selected displacement, then through the spring 25 to urge the steel ball 26 selectively in the border aperture 14 and positioning apertures 15 to form the present invention in an upright standing manner as shown in FIG. 3. The black-glass lamp 32 may be activated through the switch 23. When the black-glass lamp 32 is turned on, the ultraviolet light it emits may be used to examine skins. For instance, normal skin will become dark blue under the projection of the ultraviolet light. The skin having sunburn will have coffee color specks. The dry skin will look white or light blue. The oily skin will show orange and red colors. After the black-glass lamp 32 is turned off, the germicidal lamp 31 may be turned on through the switch 23. The ultraviolet light being generated may be used to constrain and retard the DNA of microbes, and to deprive their reproduction and breeding power. As exposing under the ultraviolet light for a prolonged period of time is harmful to human body, the present invention may include an automatic circuit to stop power supply in fifteen seconds after turning on. This will give users protection from negligent or indiscreet utilization.

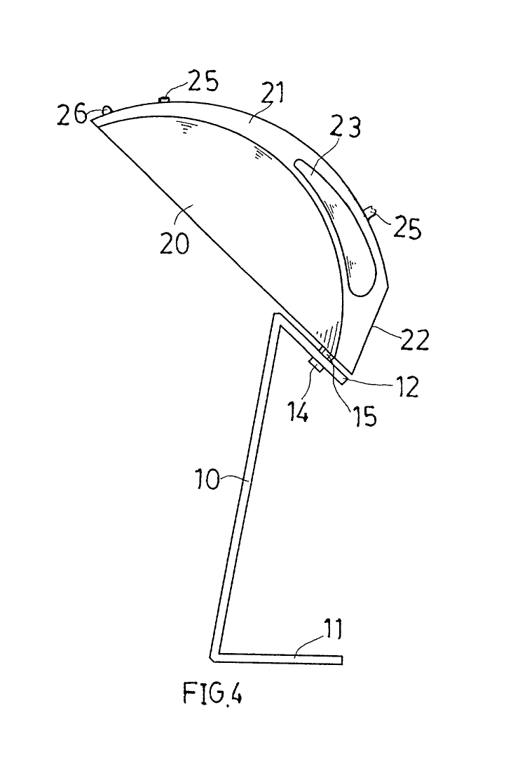

The lamp casing 20 may also be tilted an angle desired by urging the steel ball 26 through the spring 25 to a selected positioning aperture 15 as shown in FIG. 4. The lamp casing 20 forms an angle with the base frame 10. In such an occasion, the germicidal lamp 31 may be used for sterilization purpose, such as to project on foods, handkerchiefs, drinking water and the like. It is especially useful in the less developed areas where sanitary conditions are generally poor. It can greatly improve health and wellbeing of the people by sterilizing foods and water before consumption.

The base frame 10 and lamp casing 20 may also be separated. And the lamp casing 20 may be held by people's hands to project directly on articles for inspecting or sterilizing purpose.

In summary, the invention has the following advantages:

1. The invention has a black-glass lamp and germicidal lamp concurrently, and has dual effects for inspection and sterilization applications.

2. The invention is compact and portable, and may be used for skin cares anywhere and anytime with little restriction.

3. The invention is compact and portable, and may be used for sterilizing foods and articles anywhere and anytime.

4.4. The invention may be changed to different shapes and angles when in use.

What is claimed is:

1. A portable dual lamp set comprising a base frame and a lamp casing held in the base frame, the lamp casing housing two lamps therein.

2. The portable dual lamp set of claim 1, wherein the base frame includes a pedestal and a frame wall bent and extended upwards to allow the base frame standing upright, the frame wall having two side flanges located at two sides thereof, each side flange having a border aperture and a plurality of positioning apertures formed at an upper portion proximate the top rim thereof.

3. The portable dual lamp set of claim 2, wherein the frame wall is selectively a transparent board or a colored transparent board.

4. The portable dual lamp set of claim 1, wherein the lamp casing is sized to be held by hands of people, and has two lamps located therein, two slide channels located at two sides thereof, a power cord, a three-way switch and an indication light linked to the peripheral rim of the lamp casing.

5. The portable dual lamp set of claim 4, wherein the slide channel has a spring and a steel ball located in sequence in a cavity formed at an upper section proximate the top end thereof for engaging with the positioning apertures and border aperture, the cavity having a closed end.

6. The portable dual lamp set of claim 4, wherein the lamps are ultraviolet light lamps.

7. The portable dual lamp set of claim 4, wherein the lamp casing has two lamp brackets located therein for holding the lamps.

8. The portable dual lamp set of claim 7, wherein the lamp casing has a reflection hood located between the lamp brackets and the lamps for increasing light intensity of light emitting from the lamps.
